(19) United States
(12) Patent Application Publication
(10) Pub. No.: US 2023/0287334 A1
(43) Pub. Date: Sep. 14, 2023

(54) RAPIDLY DISSOLVING CELL CULTURE MEDIA POWDER AND METHODS OF MAKING THE SAME

(71) Applicant: CORNING INCORPORATED, CORNING, NY (US)

(72) Inventors: Angela Colleen Freeland, Corning, NY (US); Kai Tod Paul Jarosch, Corning, NY (US); Tyler John Lucci, Evanston, IL (US); Matthew Andrews Sevem, Wellsboro, PA (US)

(21) Appl. No.: 18/015,672

(22) PCT Filed: Jul. 1, 2021
(86) PCT No.: PCT/US2021/040099

§ 371 Date: Jan. 11, 2023

Related U.S. Application Data
(60) Provisional application No. 63/051,419, filed on Jul. 14, 2020.

(51) Int. Cl. C12N 5/00

(52) U.S. Cl. CPC C12N 5/0018

(57) ABSTRACT

A method of making a dry cell culture media powder is provided, the method including providing dry ingredients of a desired cell culture media; mixing the dry ingredients with an aqueous solution to form a paste; drying the paste to form a dehydrated media product; and milling the dehydrated media product to achieve the dry cell culture media powder of a desired particle size. A dry cell culture media powder is also provided that is rapidly dissolving, pH pre-adjusted, and pre-buffered.

This application claims the benefit of priority under 35 U.S.C. § 119 of U.S. Provisional Application Ser. No. 63/051,419 filed on Jul. 14, 2020, the content of which is relied upon and incorporated herein by reference in its entirety.

### FIELD OF THE DISCLOSURE

This disclosure general relates to cell culture media used for culturing cells, as well as methods of making such media. In particular, the present disclosure relates to rapidly dissolving cell culture media powder and methods of making such media powder.

### BACKGROUND

In the life sciences industry, cell culture media is widely used to provide the required nutrients and environment for cell growth. Cultivation of cells for the generation of therapeutics requires the use of such cell culture media which is a defined mixture of nutrients that can include carbohydrates, amino acids, lipids, vitamins, and inorganic salts. Commonly, cells are grown in a cell culture media solution to expand a cell line or to produce one or more cell by-products or compounds that are purified and sold. Characteristics and compositions of the cell culture media vary depending on the particular cellular requirements. Important parameters include osmolality, pH, and nutrient formulations. For desirable performance, the cell culture medium should provide the appropriate nutrients for cell growth at appropriate concentrations, have an appropriate pH and mechanism to maintain the appropriate pH (such as a buffer), have an appropriate osmolarity, and contain minimal amounts of contaminants, including organic, inorganic, biological, or nonbiological contaminants.

Media formulations have been used to cultivate a number of cell types including animal, plant, yeast and prokaryotic cells including bacterial cells. Cells cultivated in culture media catabolize available nutrients and produce useful biological substances such as monoclonal antibodies, hormones, growth factors, viruses and the like. Such products have therapeutic applications and, with the advent of recombinant DNA technology, cells can be engineered to produce large quantities of these products. Thus, the ability to cultivate cells is not only important for the study of cell physiology, but is also necessary for the production of useful substances which may not otherwise be obtained by cost-effective means.

Cell culture media products are manufactured and sold in both powder and liquid format, and market research suggests that powder cell culture media constitutes a larger portion of cell culture media sales worldwide than liquid cell culture media. Each of these forms has particular advantages and disadvantages. Liquid cell culture media is commonly sold as ready-to-use products, meaning that the liquid cell culture media solution does not require any preparation via the addition of chemicals before inoculation with cells. Liquid media have the disadvantages, however, that they often do require the addition of supplements (e.g., L-glutamine, serum, extracts, cytokines, lipids, vitamins, nutrients (including amino acids, nucleosides and/or nucleotides, carbon sources, one or more sugar, alcohol or other carbon containing compounds), etc.) for optimal performance in cell cultivation. Furthermore, liquid medium is often difficult to sterilize economically, since many of the components are heat labile (thus obviating the use of autoclaving, for example) and bulk liquids are not particularly amenable to penetrating sterilization methods such as gamma or ultraviolet irradiation; thus, liquid culture media are most often sterilized by filtration, which can become a time-consuming and expensive process. Furthermore, production and storage of large batch sizes (e.g., 1000 liters or more) of liquid culture media are impractical, and the components of liquid culture media often have relatively short shelf lives.

Powder cell culture media products, on the other hand, are not ready-to-use products, but instead require some preparation for use. In most cases, the procedure for preparing liquid cell culture media from a powder cell culture media product includes dissolving the powder cell culture media product in the appropriate amount of water, dissolving the buffer, and adjusting the pH of the resulting solution via addition of an acid or a base.

Although powder media is less expensive than the equivalent amount of liquid media, there are well known disadvantages to using powdered media format. For example, powder cell media products that are prepared by add-milling ingredients to form powder often exhibits slow dissolution times and a tendency to clump and/or float on the surface of water. Further, powder cell media products that are prepared by add-milling ingredients to form powder often contains a high dust content and may also exhibit poor flow characteristics. Finally, powder cell culture media products that are prepared by add-milling ingredients to form powder will tend to segregate by one or more of the many well-studied powder segregation mechanisms.

Different manufacturing methods for powder cell culture media have been developed in attempts to mitigate these difficulties associated with the use of powder cell culture media products. Many of these manufacturing methods focus on agglomerating the raw powder materials that constitute the final powder cell culture media product to achieve a final product that dissolves faster, flows more easily, and dusts less. Agglomeration is commonly done via fluidized bed agglomeration or dry agglomeration using pressure to compact the material.

Fluidized bed agglomeration generally consists of charging the agglomeration vessel with the necessary raw ingredients to formulate the product, fluidizing or suspending the raw ingredients in the vessel, and spraying said raw ingredients with solvent to promote adhesion and/or cohesion among suspended particles via mechanisms such as solid bridging. The solvent may be water, and alternative solvent, or a solution of solvent and solute(s). The agglomerated product, once dry, is then discharged from the agglomeration vessel. The agglomerated product can be packaged or further milled to achieve a smaller particle size if desired. The agglomerated product is intended to have better dissolution, flowability, and dusting characteristics compared with product made via add milling raw ingredients. Dry agglomeration generally consists of compacting dry raw ingredients into agglomerates/pellets. The agglomerated material can then be milled to achieve a smaller particle size. However, these methods do not overcome all of the difficulties of handling and using powder cell culture media.

Thus, there is a need for a powder cell culture media product that mitigates the difficulties faced by users of powdered media including slow dissolution times, clumping, floating, dusting, poor flowability, and segregation. There is also a need to a powder cell culture media that relieves media users from having to manually adjust the pH of solution after dissolving the powder product and administering the appropriate amount of buffer. Thus, a rapidly dissolving complete media powder that is pH pre-adjusted, pre-buffered, and homogeneous is needed.

### SUMMARY

According to an embodiment of this disclosure, a method of making a dry cell culture media powder is provided. The method includes providing dry ingredients of a desired cell culture media; mixing the dry ingredients with an aqueous solution to form a paste; drying the paste to form a dehydrated media product; and milling the dehydrated media product to achieve the dry cell culture media powder of a desired particle size.

As an aspect of some embodiments, the drying can include exposing the paste to a vacuum. The drying can further include exposing the paste to a vacuum until a weight of the paste stops decreasing from water evaporation. The drying can also include heating the paste.

As a further aspect of some embodiments, the mixing includes mixing the dry ingredients until the paste is homogeneous. The aqueous solution includes at least one of an acid and a base. The method can further include adjusting a composition of the aqueous solution to pre-adjust a pH of the dry cell culture media powder. The aqueous solution can include at least one of HCl and NaOH. As a further aspect, the method can include adding a buffer to the dehydrated media product. The buffer can include sodium bicarbonate.

According to an additional embodiment of this disclosure, a dry cell culture media powder is provided that is made according to the method of the above aspects and embodiments.

According to a further embodiment, a method of preparing a liquid cell culture media from a dehydrated cell culture media powder is provided. The method includes providing the dry cell culture media powder made according to the above methods. The method further includes adding the dry cell culture media powder to a solvent; and dissolving the dry cell culture media powder in the solvent to provide the liquid cell culture media. As a further aspect of embodiments, the method includes agitating the solvent while dissolving the dry cell culture media powder in the solvent.

According to aspects of some embodiments, the dissolving of the dry cell culture media is complete in about 2.5 minutes or less, in about 2 minutes or less, in about 1.5 minutes or less, in about 1 minute or less, in about 50 seconds or less, in about 45 seconds or less, in about 40 seconds or less, in about 35 seconds or less, in about 30 seconds or less, in about 25 seconds or less, in about 20 seconds or less, in about 15 seconds or less, or in about 10 seconds or less.

According to further aspects of some embodiments, during the dissolving, a maximum rate of change of a conductivity of the mixture of dry cell culture media powder and the solvent is from about 2 mS/s to about 40 mS/s, from about 4 mS/s to about 38 mS/s, from about 6 mS/s to about 36 mS/s, from about 8 mS/s to about 34 mS/s, from about 10 mS/s to about 34 mS/s, from about 12 mS/s to about 34 mS/s, from about 15 mS/s to about 34 mS/s, from about 18 mS/s to about 34 mS/s, from about 20 mS/s to about 30 mS/s, at least about 10 mS/s, at least about 15 mS/s, at least about 20 mS/s, at least about 25 mS/s, or at least about 30 mS/s.

After dissolving, the liquid cell culture media can have a pH of from about 7.0 to about 7.4. The pH of from about 7.0 to about 7.4 is achieved without adding acid or base to the dissolved dry cell culture media powder and the solvent.

According to a further embodiment of this disclosure, a dry cell culture media powder for preparing a liquid cell culture media is provided. The dry cell culture media powder includes homogenous particles containing cell culture media materials. The homogeneous particles exhibit a rate of dissolution in water measured by a rate of change of coercivity of the solution of water and the homogeneous particles, a maximum of the rate of change of coercivity being from about 2 mS/s to about 40 mS/s, from about 4 mS/s to about 38 mS/s, from about 6 mS/s to about 36 mS/s, from about 8 mS/s to about 34 mS/s, from about 10 mS/s to about 34 mS/s, from about 12 mS/s to about 34 mS/s, from about 15 mS/s to about 34 mS/s, from about 18 mS/s to about 34 mS/s, from about 20 mS/s to about 30 mS/s, at least about 10 mS/s, at least about 15 mS/s, at least about 20 mS/s, at least about 25 mS/s, or at least about 30 mS/s.

As an aspect of some embodiments, the dry cell culture media powder has homogeneous particles that are pH pre-adjusted. In addition, the homogeneous particles are pre-buffered.

Further aspects and details of embodiments of this disclosure will become clear with reference to the figures and examples discussed below.

### BRIEF DESCRIPTION OF THE DRAWINGS

FIG. 1A is a still frame from a video showing a known cell culture media powder added to a water (at time t=0).

FIG. 1B is a still frame from a video showing the cell culture media powder of FIG. 1A dissolving at time t=8 seconds.

FIG. 1C is a still frame from a video showing the cell culture media powder of FIGS. 1A and 1B at the time that it has dissolved, t=2.75 minutes.

FIG. 1D is a still frame from a video showing the cell culture media powder of FIG. 1A at the time that bicarbonate has dissolved, t=4 minutes.

FIG. 2A is a still frame from a video showing a cell culture media powder added to a water (at time t=0), according to an embodiment of the present disclosure.

FIG. 2B is a still frame from a video showing the cell culture media powder of FIG. 2A dissolving at time t=8 seconds.

FIG. 2C is a still frame from a video showing the cell culture media powder of FIGS. 2A and 2B at the time that it has dissolved, t=30 seconds.

FIG. 2D is a still frame from a video showing the cell culture media powder of FIG. 2A at the time that bicarbonate has dissolved, t=2.7 minutes.

FIG. 3 shows plots of conductivity versus time during dissolution of a conventional cell culture media solution and a cell culture media solution according to an embodiment of this disclosure.

FIG. 4 shows plots of grams of cell culture media dissolved over time for the cell culture media solutions of FIG. 3.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure relate to a powder cell culture media and methods of making such a powder cell culture media. In particular, a rapidly dissolving and homogeneous powder is provided that is complete, pH pre-adjusted, and pre-buffered. The cell culture media powder is considered “rapidly dissolving” because it can dissolve in a period of time that is significantly shorter than the period of time required for existing powder media products under specified conditions (e.g., the two are dissolved at the same concentrations when a specified amount of material of powder material is dissolved in water). The media powder of this disclosure is referred to as “complete” because the powder, when dissolved in water, offers all necessary nutrients and chemicals for maintaining an environment favorable for cell growth without any steps other than dissolution by the user. Being a complete cell culture media powder alleviates the need for chemical addition or supplementation by the customer, saving time and reducing the opportunity for batch contamination or error in material addition.

As used herein, “pH pre-adjusted” means that, when the media powder is dissolved in the appropriate amount of water, the resulting solution has a pH suitable or most desirable for the intended cell growth. An advantage of pH pre-adjustment is the savings in time for the user by eliminating the manual pH adjustment step. It also helps to reduce the opportunity for batch contamination or batch rejection that can result if the pH is adjusted incorrectly. “Pre-buffered,” as used herein, means that, in addition to being pH pre-adjusted, the solution resulting from dissolution of the media powder will have a buffering system capable of maintaining the desired pH for cell growth. Pre-buffering alleviates the need for the customer to weigh and add the appropriate amount of buffer to solution, which again saves the customer time and reduces the opportunity for batch contamination or error via material addition. Previous attempts at pH pre-adjustment have relied on the use of pH-opposing buffer salts as additives (see, e.g., International Patent Publication No. WO 02/36735). In some embodiments of the present disclosure, however, such additives can be avoided by transforming the raw ingredients of the chemically defined media into buffer materials, acids, or bases for pH pre-adjustment.

The media and methods disclosed herein differ from the existing powder cell culture media products and methods, and have many advantages over existing products and methods. Unlike conventional cell culture media powder products, embodiments of this disclosure are not simply physical mixtures of many powdered components. Instead, the powder material of the cell culture media powder of this disclosure is a single material that is chemically homogeneous in its constituent components. For example, in contrast to agglomerated powders, the powder media of the embodiments herein is not an agglomerate of individual raw ingredients. Rather, embodiments of this disclosure include a powder media with particles that are discrete and of very similar or the same size. As compared to agglomerated powders, embodiments of this disclosure also can have a greater degree of homogeneity and resistance to segregation, which is a problem seen in agglomerated powders. As used herein, “homogeneous” means that the powder will consistently yield the same components in the same concentrations when a specified amount of the powder is dissolved in the appropriate amount of water. With essentially every particle of the powder being the same or nearly the same composition, the possibility of segregation is minimized or eliminated, and the homogeneity is greatly improved or guaranteed, even for very small batches, in embodiments of powder media of this disclosure. Not only is a media powder achievable that has greater homogeneity, but also that homogeneity is able to be maintained during shipping, handling, and storage of the media powder. Unlike some alternative methods, embodiments of this disclosure do not use encapsulation of the dry cell culture media. An additional advantage of homogeneity is that a user is not forced to use the entirety of the contents of a powder product to ensure the correct proportions of physically mixed ingredients are included in the media solution. Rather, the user can use only a portion of the powder, as any portion of the media powder should have the same proportions of ingredients as any other portion or the whole. This allows for flexibility in how a user decides to use the media powder.

As discussed above, existing powder media products contain a significant amount of dust. According to embodiments of this disclosure, however, dust content is minimal. Minimal dust content provides an advantage to the customer by minimizing or eliminating the need for respirators and other safety equipment used to mitigate health risks associated with volatile powders. Furthermore, decreased dust content decreases the combustible dust hazard. Also, minimal dust content reduces the probability that the product will contaminate areas other than those designated for its use and provides for easier handling. Minimal dust content also reduces product loss to escaped dust.

Unlike common production methods for producing agglomerated cell culture media powder products, the methods of this disclosure do not use fluidized bed agglomeration or dry agglomeration. Instead, the methods disclosed herein include forming a paste with the cell culture media components, then dehydrating that paste to form a chemically homogeneous material. Due to the methods of making the powder media of this disclosure, the surface chemistry of the powder media differs from that of agglomerated particle, and the resulting powders of this disclosure achieve rapid dissolution when introduced to a solvent by the user. In particular, it is believed that a dehydration process used according to some embodiments to make the powder media can orient atoms, molecules, or functional groups of the media in a manner that quickens the rehydration process by promoting wetting, dispersion, and dissolution of the powder particles into water. This rapid dissolution has several advantages, including saving user time during the stage of mixing powder media; requiring less rigorous mixing, which can result in less air entrainment and decrease the chances of other mixing-related problems; and reducing the need to manual scrape, poke, or touch the powder that clumps onto equipment, thus reducing the risk of contamination of the media batch.

As described above, embodiments of the present disclosure provide cell culture media powders and methods of making such powder media that have advantages over existing products and methods. As a result, a powder cell culture media is provided that exhibits rapid dissolution, is pH pre-adjusted and pre-buffered, and is a complete media. Moreover, the methods of this disclosure can be applied to a wide range of cell culture media formulation. In some cases, the only adjustments to the formulation needed are the adding of acids or bases that are already used in standard cell culture media production methods. Accordingly, the methods herein are widely applicable and easily customizable.

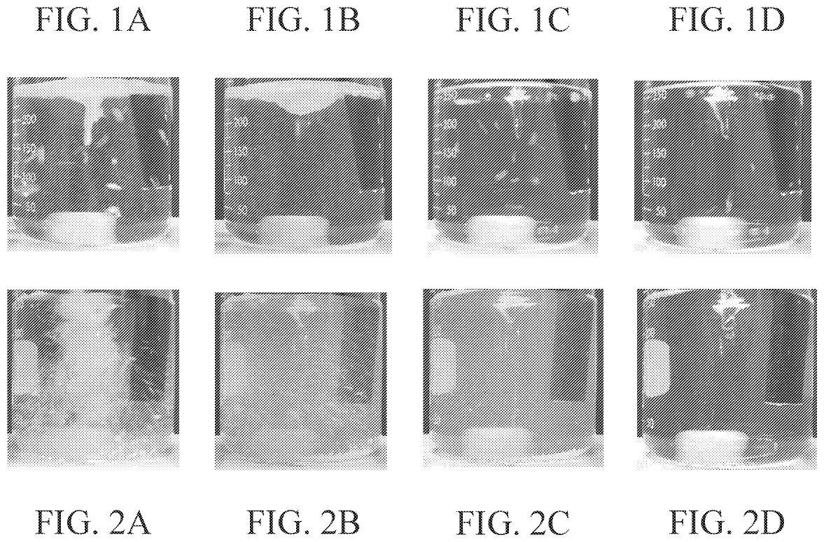

To demonstrate the rapid dissolution capability of the powder cell culture media according to embodiments of this disclosure, an existing powder cell culture media was dissolved in water and compared to the dissolution in water of a powder cell culture media of this disclosure using the same concentration and conditions. FIGS. 1A-1D show a series of still frames taken from a video for the dissolution of the pre-existing cell culture media powder product. Specifically, the pre-existing product was Dulbecco's Modified Eagle's Medium (DMEM) sold by Corning® (Corning catalog number 90-013) made according to conventional methods. FIG. 1A shows the time at which the DMEM was added to 250 mL of water in a beaker with a stir bar stirring the solution. The addition of the DMEM to the water was measured as time (t) being 0 seconds. FIG. 1B shows the solution at t=8 seconds, and it can be seen that the DMEM is dissolving. As the DMEM is dissolving, clumping is noticeable in the solution, including clumps that float on top of the solution for a relatively long period of time. At t=2.75 minutes, as shown in FIG. 1C, the media reaches the state of being completely dissolved. At t=4 minutes, the remaining bicarbonate is dissolved in the solution.

The dissolution timeline of DMEM in FIGS. 1A-1D can be compared to the dissolution of the DMEM powder media made from a paste according to embodiments of the present disclosure, which is shown in FIGS. 2A-2D. Specifically, in FIG. 2A, the media powder is added to 250 mL of water in a beaker with an active stir bar, at time (t)=0 seconds. In FIG. 2B, at t=8 seconds, the media powder can be seen dissolving. FIG. 2C shows that the media powder is completely dissolved by t=30 seconds. Further, in FIG. 2D, the bicarbonate is dissolved at t=2.7 minutes. Compared to the conventional DMEM used in FIGS. 1A-1D, the powder media used in FIGS. 2A-2D exhibits much better wettability, dispersibility, and dissolution rate. For example, no clumping or floating is visible. It is apparent in the example of FIGS. 2A-2D that many small air bubbles are released into the solution, leading to the perceived cloudiness in FIGS. 2B and 2C, particularly. However, by t=30 seconds in FIG. 2C, the powder media is completely dissolved (as evidenced by the conductivity plots discussed below), and the cloudiness is due solely to the bubbles.

In the examples of FIGS. 1A-2D, the amount of dry powder media added to the water is an amount suitable to reconstitute a liquid cell culture media suitable for culturing cells in the amount of water (i.e., 250 mL) provided. It is noted that embodiments of this disclosure are not limited to the example dissolution timeline depicted in FIGS. 2A-2D. For example, the dissolution times could be further reduced by milling the powder media. The sodium bicarbonate can also be milled before admixing to the dehydrated media to decrease its dissolution time. The DMEM used in FIGS. 1A-1D was ball milled for 3 hours, and further milling would have no impact on its dissolution time.

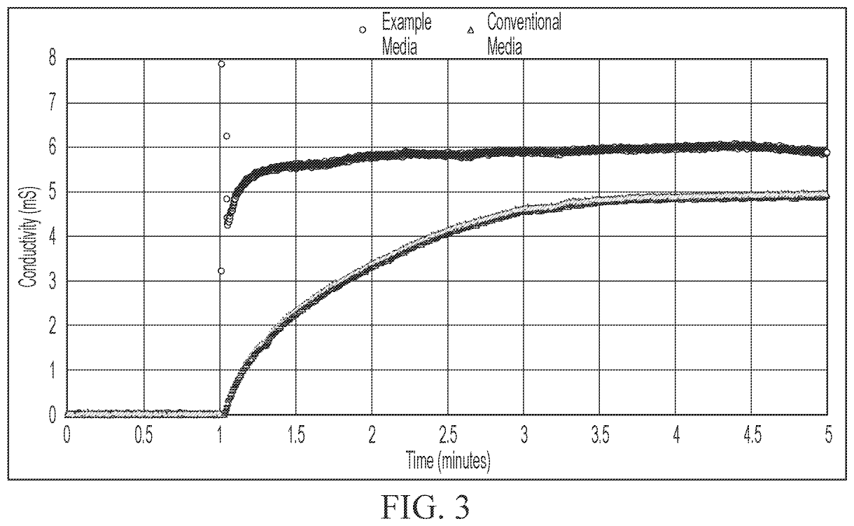

FIG. 3 is a plot of the measured conductivity (measured in milli-seimens) of the solutions in FIGS. 1A-1D and 2A-2D over time. FIG. 3 demonstrates that the conductivity of the solution containing the powder media of the present disclosure (labeled “Example Media”) increases drastically faster in the initial seconds after adding the powder to water and plateaus much sooner than the comparative DMEM sample (labeled “Conventional Media”). Because the conductivity of the solution effectively measures the degree of dissolution of the powder media into the water: as more powder dissolves, the conductivity of the solution increases. The rate of change of the conductivity and the time it takes to level-off or plateau, it reflective of the rate of dissolution of the media powder and how quickly the media powder dissolves.

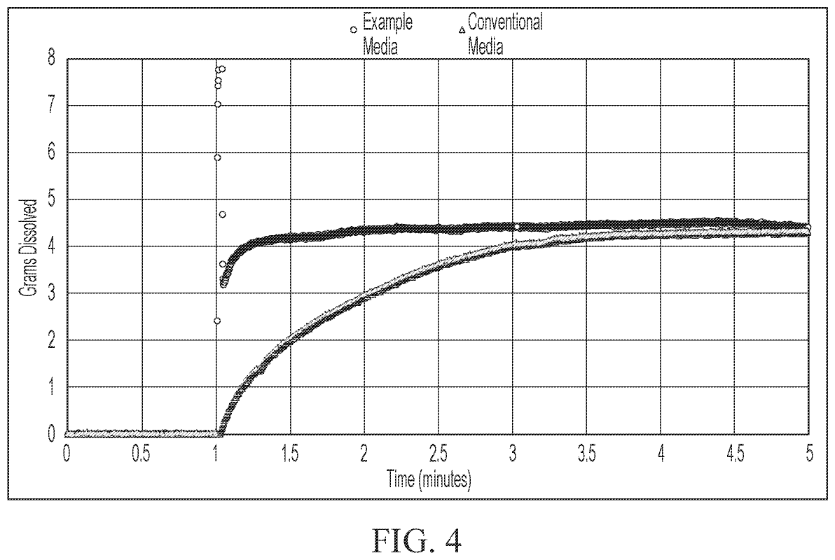

Assuming that the conductivity of the solution is proportional to the mass dissolved, the conductivity data in FIG. 3 was converted to the mass, in grams, dissolved in FIG. 4. In FIGS. 3 and 4, the powder media was added at a time corresponding to 1 minute on the x-axis of the plots. However, this time (1 minute on the x-axis of FIGS. 3 and 4) is treated as time t=0 seconds in FIGS. 1A and 2A, and the dissolution times in FIGS. 1A-2D are measured from that time going forward.

The resulting media solutions discussed above with respect to FIGS. 1A-4 were analyzed to determine the concentrations of various components using a BioProfile® Flex™ II instrument produced by Nova Biomedical. These component concentrations are shown in Table 1. Also listed in Table 1 are the specification values for each component. The Example Media according to an embodiment of the present disclosure was analyzed both before and after filtration. As shown in Table 1, the values for the Example Media Solution are within the specifications for all measured components, including pH. However, the Conventional Media Solution is outside of the outside of the specification for pH. Thus, a user would need to further adjust the pH to reach the specified range. In contrast, the Example Media is pH pre-adjusted and requires no pH adjustment after dissolving the media powder.

TABLE 1
Specification values and measured values of the Conventional
Media solution and the Example Media solution.

| | | Conventional | Example Media Solution | |
|---|---|---|---|---|
| | | Media | Before | After |
| Components | Spec | Solution | Filtration | Filtration |
| pH | 7.2 ± 0.2 | 7.791 | 7.146 | 7.376 |
| Gluc (g/L) | 4.5 | 4.41 | 4.2 | 4.3 |
| Na+ (mmol/L) | 156 | 145.1 | 137 | 134.6 |
| K+ (mmol/L) | 5.36 | 5.08 | 4.9 | 4.84 |
| Ca++ (mmol/L) | 1.8 | 1.32 | 1.26 | 1.25 |
| Osm (mOsm/kg) | 335 ± 30 | 319 | 319 | 321 |

As demonstrated by the above, embodiments of the present disclosure provide a powder cell culture media that dissolves rapidly. According to one or more embodiments, the time for total dissolution of the media powder upon being added to water is about 2.5 minutes or less, about 2 minutes or less, about 1.5 minutes or less, about 1 minute or less, about 50 seconds or less, about 45 seconds or less, about 40 seconds or less, about 35 seconds or less, about 30 seconds or less, about 25 seconds or less, about 20 seconds or less, about 15 seconds or less, or about 10 seconds or less. As shown above, the rate of dissolution of the media powder can be expressed in terms of the rate of change of the conductivity of the powder and water mixture. In one or more embodiments, during the dissolving of the powder media in water, a maximum rate of change of a conductivity of the mixture of dry cell culture media powder and the solvent is from about 2 mS/s to about 40 mS/s, from about 4 mS/s to about 38 mS/s, from about 6 mS/s to about 36 mS/s, from about 8 mS/s to about 34 mS/s, from about 10 mS/s to about 34 mS/s, from about 12 mS/s to about 34 mS/s, from about 15 mS/s to about 34 mS/s, from about 18 mS/s to about 34 mS/s, from about 20 mS/s to about 30 mS/s, at least about 10 mS/s, at least about 15 mS/s, at least about 20 mS/s, at least about 25 mS/s, or at least about 30 mS/s.

As discussed above, one or more embodiments of this disclosure provide for cell culture media powder, and methods of making such powder, in the form of DMEM. However, embodiments of this disclosure can be applied to any various media formulations.

Examples of animal cell culture media that may be prepared according to embodiments of the present disclosure include, but are not limited to, DMEM, RPMI-1640, MCDB 131, MCDB 153, MDEM, IMDM, MEM, M199, McCoy's 5A, Williams' Media E, Leibovitz's L-15 Medium, Grace's Insect Medium, IPL-41 Insect Medium, TC-100 Insect Medium, Schneider's Drosophila Medium, Wolf & Quimby's Amphibian Culture Medium, F10 Nutrient Mixture, F12 Nutrient Mixture, and cell-specific serum-free media (SFM) such as those designed to support the culture of keratinocytes, endothelial cells, hepatocytes, melanocytes, CHO cells, 293 cells, PerC6, hybridomas, hematopoetic cells, embryonic cells, neural cells etc. Other media, media supplements and media subgroups suitable for preparation by embodiments of this disclosure are available commercially (e.g., from Life Technologies, Inc., Rockville, Md.; and Sigma, St. Louis, Mo.). Formulations for these media, media supplements and media subgroups, as well as many other commonly used cell culture media, media supplements and media subgroups are well-known in the art and may be found, for example in the GIBCO/BRL Catalogue and Reference Guide (Life Technologies, Inc., Rockville, Md.) and in the Sigma Animal Cell Catalogue (Sigma, St. Louis, Mo.).

Examples of plant cell culture media that may be prepared according to embodiments of the present disclosure include, but are not limited to, Anderson's Plant Culture Media, CLC Basal Media, Gamborg's Media, Guillard's Marine Plant Culture Media, Provasoli's Marine Media, Kao and Michayluk's Media, Murashige and Skoog Media, McCown's Woody Plant Media, Knudson Orchid Media, Lindemann Orchid Media, and Vacin and Went Media. Formulations for these media, which are commercially available, as well as for many other commonly used plant cell culture media, are well-known in the art and may be found for example in the Sigma Plant Cell Culture Catalogue (Sigma, St. Louis, Mo.).

Examples of bacterial cell culture media that may be prepared according to embodiments of the present disclosure include, but are not limited to, Trypticase Soy Media, Brain Heart Infusion Media, Yeast Extract Media, Peptone-Yeast Extract Media, BeefInfusion Media, Thioglycollate Media, Indole-Nitrate Media, MR-VP Media, Simmons Citrate Media, CTA Media, Bile Esculin Media, Bordet-Gengou Media, Charcoal Yeast Extract (CYE) Media, Mannitol-salt Media, MacConkey's Media, Eosin-methylene blue (EMB) media, Thayer-Martin Media, Salmonella-Shigella Media, and Urease Media. Formulations for these media, which are commercially available, as well as for many other commonly used bacterial cell culture media, are well-known in the art and may be found for example in the DIFCO Manual (DIFCO, Norwood, Mass.) and in the Manual of Clinical Microbiology (American Society for Microbiology, Washington, D.C.).

Examples of fungal cell culture media, particularly yeast cell culture media, that may be prepared according to embodiments of the present disclosure include, but are not limited to, Sabouraud Media and Yeast Morphology Media (YMA). Formulations for these media, which are commercially available, as well as for many other commonly used yeast cell culture media, are well-known in the art and may be found for example in the DIFCO Manual (DIFCO, Norwood, Mass.) and in the Manual of Clinical Microbiology (American Society for Microbiology, Washington, D.C.).

As the skilled artisan will appreciate, any of the above media may also include one or more additional components, such as indicating or selection agents (e.g., dyes, antibiotics, amino acids, enzymes, substrates and the like), filters (e.g., charcoal), salts, polysaccharides, ions, detergents, stabilizers, and the like.

According to one or more embodiments, the powder media may include one or more amino acids, vitamins, inorganic salts, and other components. For example, the powder media can include glycine, L-alanine, L-arginine, L-arginine hydrochloride, L-asparagine-H₂O, L-aspartic acid, L-cysteine hydrochloride-H₂O, L-cystine 2HCl, L-glutamine, L-glutamic acid, L-histidine, L-histidine hydrochloride-H₂O, L-isoleucine, L-leucine, L-lysine hydrochloride, L-methionine, L-phenylalanine, L-proline, L-serine, L-threonine, L-tryptophan, L-tyrosine disodium salt dihydrate, L-valine, ascorbic acid, biotin, choline chloride, D-calcium pantothenate, folic acid, niacinamide, pyridoxal hydrochloride, riboflavin, vitamin B12, thiamine hydrochloride, i-inositol, calcium chloride (CaCl₂), magnesium sulfate (MgSO₄), potassium chloride (KCl), sodium bicarbonate (NaHCO₃), sodium chloride (NaCl), sodium phosphate monobasic (NaH₂PO₄—H₂O), D-glucose (Dextrose), lipoic acid, HEPES, sodium pyruvate, and phenol red.

Methods of producing the powder according to embodiments of this disclosure will now be described. According to one or more embodiment, the method includes proving raw powder ingredients, optionally in an open vessel, and adding an amount of solvent to the powder. The raw ingredients can include amino acids, proteins, and any of a number of ingredients used in cell culture media. As discussed above, the components can be varied as desired to result in a particular cell culture media (e.g., DMEM powder). The solvent can be water, acid (e.g., HCl), base (e.g., NaOH), or another solution of water-soluble material. The powder and solvent combination are then mixed to achieve homogeneity. Preferably, the amount of solvent added is relatively small, such that the resulting mixture forms a paste, rather than a liquid. The homogeneous mixture is then exposed to an environment under vacuum. For example, the vessel containing the mixture can be placed in a vacuum chamber. The mixture is dehydrated by the vacuum. Optionally, buffer is added to the dehydrated powder product. The resulting dehydrated product is then milled to achieve a desired particle size.

As an aspect of some embodiments, the mixture is temperature controlled while in vacuum. For example, the mixture can be placed on a heat source within the vacuum chamber. According to some embodiments, the mixture is not frozen while under vacuum. In other words, lyophilization is not used (i.e., freeze drying). In some embodiments, vacuum is pulled at negative 28 inches of mercury. The amount of dehydration of the mixture can vary, but in some embodiments the product is dehydrated until weight loss due to water evaporation is no longer observed.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a method of making a dry cell culture media powder comprising the steps of: providing dry ingredients of a desired cell culture media; mixing the dry ingredients with an aqueous solution to form a paste; drying the paste to form a dehydrated media product; and milling the dehydrated media product to achieve the dry cell culture media powder of a desired particle size.

Aspect 2 pertains to the method of Aspect 1, wherein the drying comprises exposing the paste to a vacuum.

Aspect 3 pertains to the method of Aspect 2, wherein the drying comprises exposing the paste to a vacuum until a weight of the paste stops decreasing from water evaporation.

Aspect 4 pertains to the method of any one of Aspects 1-3, wherein the drying comprising heating the paste.

Aspect 5 pertains to the method of any of Aspects 1-4, wherein the mixing comprising mixing the dry ingredients until the paste is homogeneous.

Aspect 6 pertains to the method of any of Aspects 1-5, wherein the aqueous solution comprises at least one of an acid and a base.

Aspect 7 pertains to the method of Aspect 6, the method further comprising adjusting a composition of the aqueous solution to pre-adjust a pH of the dry cell culture media powder.

Aspect 8 pertains to the method of any of Aspects 1-7, wherein the aqueous solution comprises at least one of HCl and NaOH.

Aspect 9 pertains to the method of any of Aspects 1-8, further comprising adding a buffer to the dehydrated media product.

Aspect 10 pertains to the method of Aspect 9, wherein the buffer comprises sodium bicarbonate.

Aspect 11 pertains to a dry cell culture media powder made according to the method of any one of Aspects 1-10.

Aspect 12 pertains to a method of preparing a liquid cell culture media from a dehydrated cell culture media powder, the method comprising: providing the dry cell culture media powder made according to the method of any one of Aspects 1-10; adding the dry cell culture media powder to a solvent; and dissolving the dry cell culture media powder in the solvent to provide the liquid cell culture media.

Aspect 13 pertains to the method of Aspect 12, further comprising agitating solvent while dissolving the dry cell culture media powder in the solvent.

Aspect 14 pertains to the method of Aspect 12 or Aspect 13, wherein the dissolving of the dry cell culture media is complete in about 2.5 minutes or less, in about 2 minutes or less, in about 1.5 minutes or less, in about 1 minute or less, in about 50 seconds or less, in about 45 seconds or less, in about 40 seconds or less, in about 35 seconds or less, in about 30 seconds or less, in about 25 seconds or less, in about 20 seconds or less, in about 15 seconds or less, or in about 10 seconds or less.

Aspect 15 pertains to the method of any of Aspects 12-14, wherein, during the dissolving, a maximum rate of change of a conductivity of the mixture of dry cell culture media powder and the solvent is from about 2 mS/s to about 40 mS/s, from about 4 mS/s to about 38 mS/s, from about 6 mS/s to about 36 mS/s, from about 8 mS/s to about 34 mS/s, from about 10 mS/s to about 34 mS/s, from about 12 mS/s to about 34 mS/s, from about 15 mS/s to about 34 mS/s, from about 18 mS/s to about 34 mS/s, from about 20 mS/s to about 30 mS/s, at least about 10 mS/s, at least about 15 mS/s, at least about 20 mS/s, at least about 25 mS/s, or at least about 30 mS/s.

Aspect 16 pertains to the method of any of Aspects 12-15, wherein, after dissolving, the liquid cell culture media has a pH of from about 7.0 to about 7.4.

Aspect 17 pertains to the method of Aspect 16, wherein the pH of from about 7.0 to about 7.4 is achieved without adding acid or base to the dissolved dry cell culture media powder and the solvent.

Aspect 18 pertains to a dry cell culture media powder for preparing a liquid cell culture media, comprising: homogenous particles comprising cell culture media materials, wherein the homogeneous particles comprise a rate of dissolution in water measured by a rate of change of coercivity of the solution of water and the homogeneous particles, a maximum of the rate of change of coercivity being from about 2 mS/s to about 40 mS/s, from about 4 mS/s to about 38 mS/s, from about 6 mS/s to about 36 mS/s, from about 8 mS/s to about 34 mS/s, from about 10 mS/s to about 34 mS/s, from about 12 mS/s to about 34 mS/s, from about 15 mS/s to about 34 mS/s, from about 18 mS/s to about 34 mS/s, from about 20 mS/s to about 30 mS/s, at least about 10 mS/s, at least about 15 mS/s, at least about 20 mS/s, at least about 25 mS/s, or at least about 30 mS/s.

Aspect 19 pertains to the dry cell culture media powder of Aspect 18, wherein the homogeneous particles are pH pre-adjusted.

Aspect 20 pertains to the dry cell culture media powder of Aspect 18 or Aspect 19, wherein the homogeneous particles are pre-buffered.

### Definitions

As used herein, “cell culture media” and “media” are used interchangeably and refer to a substance used during the culturing of cells to provide an environment and/or nutrients required by the cells. The cell culture medium may be a complete formulation, i.e., a cell culture medium that requires no supplementation to culture cells, may be an incomplete formulation, i.e., a cell culture medium that requires supplementation or may be a medium that may supplement an incomplete formulation or in the case of a complete formulation, may improve culture or culture results. The terms “cell culture medium,” “culture medium,” or “medium” (and in each case plural media) refer to unconditioned cell culture media that has not been incubated with cells, unless indicated otherwise from the context. As such, the terms “cell culture medium,” “culture medium,” or “medium” (and in each case plural media) are distinguished from “spent” or “conditioned” medium, which may contain many of the original components of the medium, as well as a variety of cellular metabolites and secreted proteins. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems or whole organisms, for which the terms “tissue culture,” “organ culture,” “organ system culture” or “organotypic culture” may occasionally be used interchangeably with the term “cell culture.”

The term “cultivation” as used in this application refers to the maintenance of cells in an artificial environment under conditions favoring growth, differentiation, or continued viability, in an active or quiescent state, of the cells. Thus, “cultivation” may be used interchangeably with “cell culture” or any of its synonyms described above.

The term “powder” or “powdered” as used in this application refers to a composition that is present in granular form, which may or may not be complexed or agglomerated with a solvent such as water or serum. The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated. “Powder cell culture media” and “cell culture media powder” are used interchangeably and refer to a cell culture media in powdered form.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A method of making a dry cell culture media powder comprising the steps of:
providing dry ingredients of a desired cell culture media;
mixing the dry ingredients with an aqueous solution to form a paste;
drying the paste to form a dehydrated media product; and
milling the dehydrated media product to achieve the dry cell culture media powder of a desired particle size.

2. The method of claim 1, wherein the drying comprises exposing the paste to a vacuum.

3. The method of claim 2, wherein the drying comprises exposing the paste to a vacuum until a weight of the paste stops decreasing from water evaporation.

4. The method of claim 1, wherein the drying comprising heating the paste.

5. The method of claim 1, wherein the mixing comprising mixing the dry ingredients until the paste is homogeneous.

6. The method of claim 1, wherein the aqueous solution comprises at least one of an acid and a base.

7. The method of claim 6, the method further comprising adjusting a composition of the aqueous solution to pre-adjust a pH of the dry cell culture media powder.

8. The method of claim 1, wherein the aqueous solution comprises at least one of HCl and NaOH.

9. The method of claim 1, further comprising adding a buffer to the dehydrated media product.

10. The method of claim 9, wherein the buffer comprises sodium bicarbonate.

11. A dry cell culture media powder made according to the method of claim 1.

12. A method of preparing a liquid cell culture media from a dehydrated cell culture media powder, the method comprising:
providing the dry cell culture media powder made according to the method of claim 1;
adding the dry cell culture media powder to a solvent; and
dissolving the dry cell culture media powder in the solvent to provide the liquid cell culture media.

13. The method of claim 12, further comprising agitating solvent while dissolving the dry cell culture media powder in the solvent.

14. The method of claim 12, wherein the dissolving of the dry cell culture media is complete in about 2.5 minutes or less, in about 2 minutes or less, in about 1.5 minutes or less, in about 1 minute or less, in about 50 seconds or less, in about 45 seconds or less, in about 40 seconds or less, in about 35 seconds or less, in about 30 seconds or less, in about 25 seconds or less, in about 20 seconds or less, in about 15 seconds or less, or in about 10 seconds or less.

15. The method of claim 12, wherein, during the dissolving, a maximum rate of change of a conductivity of the mixture of dry cell culture media powder and the solvent is from about 2 mS/s to about 40 mS/s, from about 4 mS/s to about 38 mS/s, from about 6 mS/s to about 36 mS/s, from about 8 mS/s to about 34 mS/s, from about 10 mS/s to about 34 mS/s, from about 12 mS/s to about 34 mS/s, from about 15 mS/s to about 34 mS/s, from about 18 mS/s to about 34 mS/s, from about 20 mS/s to about 30 mS/s, at least about 10 mS/s, at least about 15 mS/s, at least about 20 mS/s, at least about 25 mS/s, or at least about 30 mS/s.

16. The method of claim 12, wherein, after dissolving, the liquid cell culture media has a pH of from about 7.0 to about 7.4.

17. The method of claim 16, wherein the pH of from about 7.0 to about 7.4 is achieved without adding acid or base to the dissolved dry cell culture media powder and the solvent.

18. A dry cell culture media powder for preparing a liquid cell culture media, comprising:
homogenous particles comprising cell culture media materials,
wherein the homogeneous particles comprise a rate of dissolution in water measured by a rate of change of coercivity of the solution of water and the homogeneous particles, a maximum of the rate of change of coercivity being from about 2 mS/s to about 40 mS/s, from about 4 mS/s to about 38 mS/s, from about 6 mS/s to about 36 mS/s, from about 8 mS/s to about 34 mS/s, from about 10 mS/s to about 34 mS/s, from about 12 mS/s to about 34 mS/s, from about 15 mS/s to about 34 mS/s, from about 18 mS/s to about 34 mS/s, from about 20 mS/s to about 30 mS/s, at least about 10 mS/s, at least about 15 mS/s, at least about 20 mS/s, at least about 25 mS/s, or at least about 30 mS/s.

19. The dry cell culture media powder of claim 18, wherein the homogeneous particles are pH pre-adjusted.

20. The dry cell culture media powder of claim 18, wherein the homogeneous particles are pre-buffered.
